(19) United States
(12) Patent Application Publication
(10) Pub. No.: US 2005/0070951 A1
(43) Pub. Date: Mar. 31, 2005

(54) Medical device for explantation

(76) Inventor: Pascal Paganon, Villeurbanne (FR)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 10/500,242

(22) PCT Filed: Dec. 23, 2002
(86) PCT No.: PCT/FR02/04537

(30) Foreign Application Priority Data
Dec. 28, 2001 (FR) 01/17099

(51) Int. Cl. A61M 29/00

(52) U.S. Cl. 606/198

(57) ABSTRACT

The invention relates to a medical device (1) for explanting a flexible pouch (P) containing a fluid, which pouch (P) comprises an envelope (E) having an inside face (F), said device comprising at least perforation means (2C) for perforating the pouch (P) and securing means for securing the device to the pouch (P) so as to explant it, said medical device being characterized in that said perforation means (2C) make it possible to form an orifice in the flexible pouch (P) so as to enable the securing means to pass through, which means are formed by anchor means (12) acting from the inside of the pouch (P) on a portion of said inside face (F) of the envelope to generate bearing engagement sufficient to enable the pouch to be explanted. An endoscopic medical device for explantation.

### CROSS-REFERENCE TO RELATED APPLICATIONS

This application is a national stage application of International Application No. PCT/FR02/04537, filed Dec. 23, 2002, which claims priority to FR 01/17099, filed Dec. 28, 2001, both applications of which are hereby incorporated by reference in their entirety into the present application.

### TECHNICAL FIELD

The present invention relates to the technical field of medical devices designed to be inserted into the operator channel of an endoscope or of any other equivalent instrument for performing various manipulations inside organs or cavities of the body of a patient.

The present invention relates to an endoscopic medical device for retrieval, in particular of any foreign body resembling a flexible and perforatable pouch situated in the stomach. Such retrieval devices may also be referred to as “explantation devices”.

### PRIOR ART

In patients suffering from extremely severe obesity (morbid obesity), i.e. in patients who are overweight by at least 50 kilograms (kg), for example, relative to their ideal weight, and in patients who are overweight by at least 40% of their ideal weight, medical surgery is absolutely necessary in order not only to avoid a series of health problems resulting from such obesity, but also to avoid certain or imminent death in such patients.

Devices are known that make it possible to treat morbid obesity, and that are constituted by a band which, by being surgically implanted and closed as a ring around the stomach, makes it possible to limit the quantity of food absorbed. Implanting that type of device requires the patient to undergo major surgery.

Intra-gastric devices of the balloon type are also known that are relatively easy to implant since such implantation can be performed by gastroenterologists or surgeons via the natural passageway and under endoscopic control, under local or general anesthetic.

Such an intra-gastric balloon, placed in the stomach of an obese person, reduces the working volume of the stomach, thereby giving the obese person the feeling of being sated even though they have absorbed quantities of food that are smaller than the quantities of food necessary for obtaining the same feeling of being sated without the intra-gastric balloon. Therefore, implanted intra-gastric balloons reduce the appetite of obese people, and induce significant weight loss in them.

Commonly, an intra-gastric balloon is formed by a pouch made of a flexible and expandable material, and is inserted, while empty, endoscopically into the stomach. It is then filled with a fluid (gas or liquid) that is sterile or otherwise. When it is full, the balloon is thus too large to pass into the intestine, and it thus floats freely in the stomach.

However, removing the balloon, which needs to be done after a few weeks or a few months, is problematic. The process commonly used for explanting the balloon is made up of the following steps, in chronological order:
the practitioner inserts a hollow catheter containing a guide wire into the stomach;
the practitioner punctures the balloon with the catheter equipped with its guide wire;
the guide wire is withdrawn from the catheter in order to leave a passageway for the fluid;
the fluid contained in the intra-gastric balloon is sucked out through the catheter, until the balloon is empty;
the catheter is withdrawn from the endoscope;
the practitioner inserts a second catheter equipped with a snare;
the practitioner takes hold of the deflated balloon with the snare; and
the assembly formed by the endoscope, by the catheter equipped with the snare, and by the deflated balloon is extracted from the stomach and from the body of the patient.

That process suffers from the drawback of taking a long time, in particular because it implements two different tools (a first catheter for puncture and suction purposes and a second catheter equipped with a snare).

The operation time is thus made longer, which naturally goes against the interest of the patient.

The use of two different tools is also a factor reducing patient safety because, between the time when the first catheter is removed and the time when the deflated balloon is caught with the snare, said deflated balloon floats freely in the stomach and can pass into the intestines and become lodged therein, thereby giving rise to an intestinal obstruction, which is a serious complication that can require surgery.

In addition capturing the deflated balloon with the snare can sometimes be very difficult, requiring very considerable dexterity from the partitioner, which again is a factor of risk and of discomfort for the patient.

Finally, the use of two tools, and the ensuing lengthening of the operation time give rise to relatively large costs for the explantation operation.

Document EP-0 205 228 describes an endoscopic device provided firstly with a retractable needle making it possible to puncture an intra-gastric balloon filled with gas, and secondly with a snare or jaw for taking hold of the balloon for the purpose of explanting it.

However, that device is not suitable for balloons containing liquid because it is not provided with any means for removing the fluid to the outside of the body of the patient. Naturally, it is unthinkable for a balloon filled with liquid to be emptied directly into the stomach of the patient, if only because proliferation of bacteria might have taken place in the liquid.

In addition, as mentioned above, capturing the balloon with a snare, or indeed with jaws, can be very difficult, which increases the risks for the patient, and which can lengthen the time and thus the cost of the operation. An endoscopic device provided with a jaw also suffers from a risk of trauma for the wall of the stomach.

### SUMMARY OF THE INVENTION

An object of the invention is therefore to propose a novel medical device for explanting a flexible pouch, which devices makes it possible to remedy the various above-mentioned drawbacks and is suitable for performing explantation that is fast, safe, and inexpensive, and during which the work of the practitioner is facilitated.

Another object of the invention is to provide a novel explantation device that is atraumatic and that operates simply and reliably.

Another object of the invention is to provide a novel medical device for explanting a flexible pouch, which device is suitable for performing a snare function as is well known to practitioners.

Another object of the invention is to provide a novel explantation device that is particularly easy and inexpensive to manufacture.

Another object of the invention is to provide a novel explantation device to be used once only or more than once.

The objects of the invention are achieved by means of a medical device for explanting a flexible pouch containing a fluid, which pouch comprises an envelope having an inside face, said device comprising at least perforation means for perforating the pouch and securing means for securing the device to the pouch so as to explant it, said medical device being characterized in that said perforation means make it possible to form an orifice in the flexible pouch so as to enable the securing means to pass through, which means are formed by anchor means acting from the inside of the pouch on a portion of said inside face of the envelope to generate bearing engagement sufficient to enable the pouch to be explanted.

### BRIEF DESCRIPTION OF THE DRAWINGS

Other objects and advantages of the invention will appear more clearly on reading the following description, with reference to the accompanying drawings which are given merely by way on non-limiting illustration, and in which:

FIG. 1 is a diagrammatic perspective view of an embodiment of an explantation device of the invention in the deactivated position, i.e. when the anchor means are not capable of generating bearing engagement against the inside face of the envelope;

FIG. 2 is a diagrammatic perspective view showing the device of FIG. 1 in the anchor position, i.e. when the anchor means are activated and capable of generating bearing engagement against the inside face of the envelope;

FIG. 3 is a fragmentary longitudinal section view of a detail of the device shown in FIGS. 1 and 2;

FIG. 4 is a section view of a detail of the device shown in FIGS. 1 to 3;

FIG. 5 is a diagrammatic section view of a detail of a variant embodiment of a medical device of the invention for explanting a flexible pouch;

FIG. 6 is a diagrammatic perspective view of a detail of a variant embodiment of a medical explantation device of the invention; and

FIG. 7 is a diagrammatic perspective view of a detail of a variant embodiment of a medical explantation device of the invention.

### BEST MANNER OF IMPLEMENTING THE INVENTION

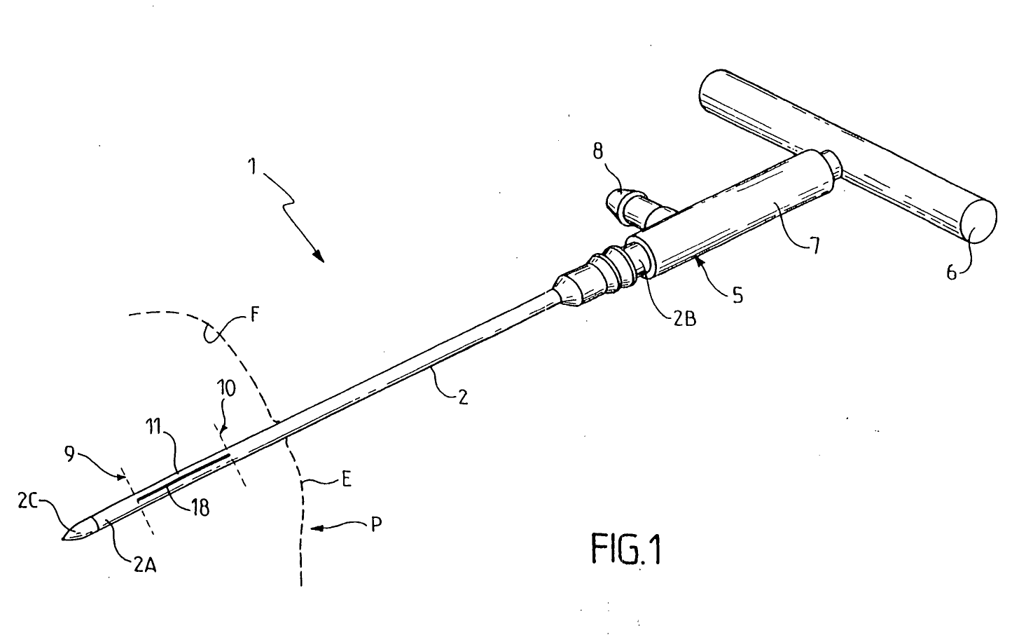

FIGS. 1, 2, 6, and 7 show an embodiment of a medical device 1 for explanting a flexible pouch P containing a fluid, which pouch P comprises an envelope E having an inside wall F. The device comprises at least perforation means 2C for perforating the pouch and securing means for securing the device to the pouch so as to explant it. According to an important characteristic of the invention, the perforation means 2C make it possible to form an orifice in the flexible pouch so as to enable the securing means to pass through, which means are formed by anchor means 12 acting from the inside of the pouch on a portion of the inside face of the envelope to generate bearing engagement sufficient to enable the pouch to be explanted. In this way, the force exerted by the user on the medical explantation device 1 to extract it from the body of the patient is transmitted in full to the inside wall of the flexible pouch so that said pouch goes with the medical explantation device 1 as it is being brought out of the stomach and out of the body of the patient.

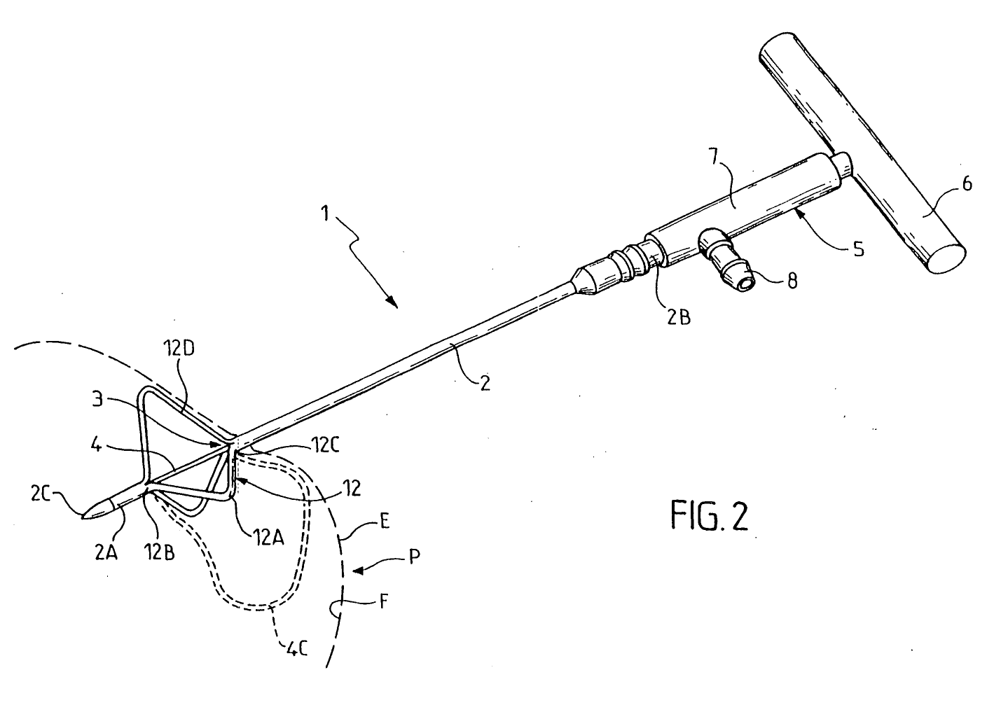

Advantageously, and as shown in FIGS. 1, 2, 6, and 7, the anchor means 12 are capable of being deployed, their deployment being controlled by a control member 6 (not shown in FIGS. 6 and 7). Deploying the anchor means 12 consists in said anchor means going from a state that can be termed “deactivated”, in which they occupy a small volume, as shown in FIG. 1, to an activated state, as shown in FIGS. 2, 4, 6, and 7, in which the anchor means 12 occupy a larger volume, so that they come to bear against a portion of the inside face F of the envelope E of the pouch P, so as to entrain said face when the medical explantation device 1 is withdrawn from the body of the patient. Such a result can be obtained, for example, with anchor means 12 in the deployed state capable of having a bearing area over which they are in bearing engagement against the inside face F of the envelope E that is large enough to enable the pouch to be explanted. Naturally, it is possible to achieve a similar result with a smaller bearing area, which can, for example, consist merely in bearing points, as obtained with the embodiment of the device 1 shown in FIG. 7, the bearing points nevertheless being distributed appropriately for transmitting the explantation movement to the pouch, without any risk of the pouch being damaged to the point of slipping off the anchor means 12.

Advantageously, the medical explantation device 1 of the invention for explanting a flexible pouch P is further provided with fluid removal means 3, 8 for removing the fluid contained inside the pouch to the outside of the body of the patient. The medical explantation device 1 of the invention can thus preferably make it possible to suck out the fluid contained inside the pouch while said pouch is still contained inside the body of the patient.

In a first variant embodiment of the medical explantation device shown in FIG. 6, the anchor means 12 are formed by a structure forming a hinged truss and comprising at least one anchor arm 12D, said structure having a front termination 20 and a back termination 21, which terminations are caused to move closer together in controlled manner by the control member (not shown) in order to cause the at least one anchor arm 12D to be deployed.

The anchor arm 12D may advantageously be made up of two elongate pieces in the form of beams 30, 31, each of the beams having two ends 30A, 30B, 31A, 31B. One end 30A, 31A of a respective one of the beams is pivotally coupled to the front termination 20, and one end 31A, 30A of the other beam is pivotally coupled to the back termination 21, the other ends 30B, 31B of the beams being pivotally coupled to each other. The front termination 20 and the back termination 21 are mounted to slide so that, when they are spaced apart from each other to the maximum extent, the beams 30, 31 are in alignment, and when said terminations 20, 21 are brought closer together, the beams 30, 31 move pivotally so as to form an anchor arm 12D extending transversely to the axis of sliding of the front and back terminations 20, 21.

Naturally, it is possible to imagine structures forming different hinged trusses without going beyond the ambit of the invention. For example, a device of the umbrella mechanism type, as shown diagrammatically in FIG. 7 could be quite suitable.

In a preferred variant of an explantation device 1 of the invention, said device comprises, as shown in FIGS. 1 and 2, a hollow tube 2 that has a distal portion 2A provided with the perforation means 2C and a proximal portion 2B, said distal portion 2A and said proximal portion 2B defining between them an internal volume 3.

The hollow tube 2 is preferably made of a material offering a good compromise between flexibility and rigidity so that it can be used as a hollow catheter, and, in that capacity, be inserted into an endoscope (or any other similar instrument) provided with an operator channel having a diameter preferably greater than 2.8 millimeters (mm). Naturally, that value is non-limiting, and the explantation device of the invention can be used with an operator channel of any diameter.

The perforation means 2C can advantageously, and as shown in FIGS. 1, 2, and 4, be formed by the tip of a separate piece threaded into the distal portion 2A of the hollow tube 2 so as to form a uniform extension to the distal portion 2A. Naturally, without going beyond the ambit of the invention, other embodiments can be imagined for the perforation means, such as, for example, beveling the distal portion 2A or installing a needle.

The medical explantation device of said preferred variant embodiment of the invention also has a guide wire 4 disposed in the internal volume 3 of the hollow tube 2 and having a front end 4A and a back end 4B.

The term “guide wire” is used to designate a cable of any section that offers a good compromise between rigidity and flexibility, so as to stiffen the hollow tube 2, in particular with a view to performing the perforation to be performed at the end 2A, while being flexible enough to form a snare loop.

In this preferred embodiment of the invention, the medical explantation device 1 is also provided with an external connector 5 mounted at the proximal portion 2B via a sleeve 7 having a cavity 7A, and including the control member 6 organized to make it possible for a user to exert at least traction and compression forces on the guide wire 4. For this purpose, said control member 6 is mounted to move relative to the sleeve 7, the cavity 7A of said sleeve communicating with the outside via one or more tubular end-pieces 8. The front end 4A and the back end 4B of the guide wire 4 are secured respectively to the distal portion 2A of the hollow tube 2 and to the control member 6.

According to an essential characteristic of the explantation device of this preferred variant of the invention, the hollow tube 2 includes at least one weak segment 18 extending between a front section 9 and a back section 10 of the hollow tube 2, over a length sufficient to define with said sections 9, 10 a portion 11 of hollow tube 2 that, when the user exerts traction on the back end 4B via the control member 6, thereby causing the hollow tube 2 to be compressed axially, tends to undergo buckling causing it to be deformed towards the outside of the internal volume 3 along said at least one weak segment 18 so that said portion 11 forms the anchor means 12 suitable for being deployed.

Buckling of a part is a well-known phenomenon of mechanical instability that results in deformation (which can merely be bending) of the part under the effect of a longitudinal compression force to which it is subjected at its ends.

However, the phenomenon of buckling generally occurs only for parts such as plates or beams in which at least one of the dimensions is considerably smaller than the other two dimensions (e.g., for a plate, in which its thickness is much smaller than its length and its width).

For example, if consideration is given to a part having the shape of a beam (i.e. in which its length is much larger than its width and its thickness), it is known that the appearance of buckling is mainly a function of the intensity of the longitudinal compression force applied, of the intrinsic properties of the material of which the part is made (in particularly its elasto-plastic properties), and of the length of the part.

It can thus be understood that, when buckling is to be achieved by applying the compression force manually, said force cannot exceed a certain limit, which corresponds to the limit of the strength of the user. Therefore, if the buckling is to be achieved easily, it is necessary for the part to be sufficiently long and to be made of material that is sufficiently elastic. Since the deformations caused by the phenomenon of buckling are of large amplitude, it is also necessary for the material of which the part is made to have good plastic properties, and in particular to be ductile so that the part does not break.

The purpose of the weak segment(s) 18 with which the hollow tube 2 of the medical explantation device 1 of the preferred variant of the invention is provided is to define a portion 11 of the hollow tube 2 that satisfies the buckling conditions, i.e. that is made up of one or more elements in which length is much larger than at least thickness (and optionally width).

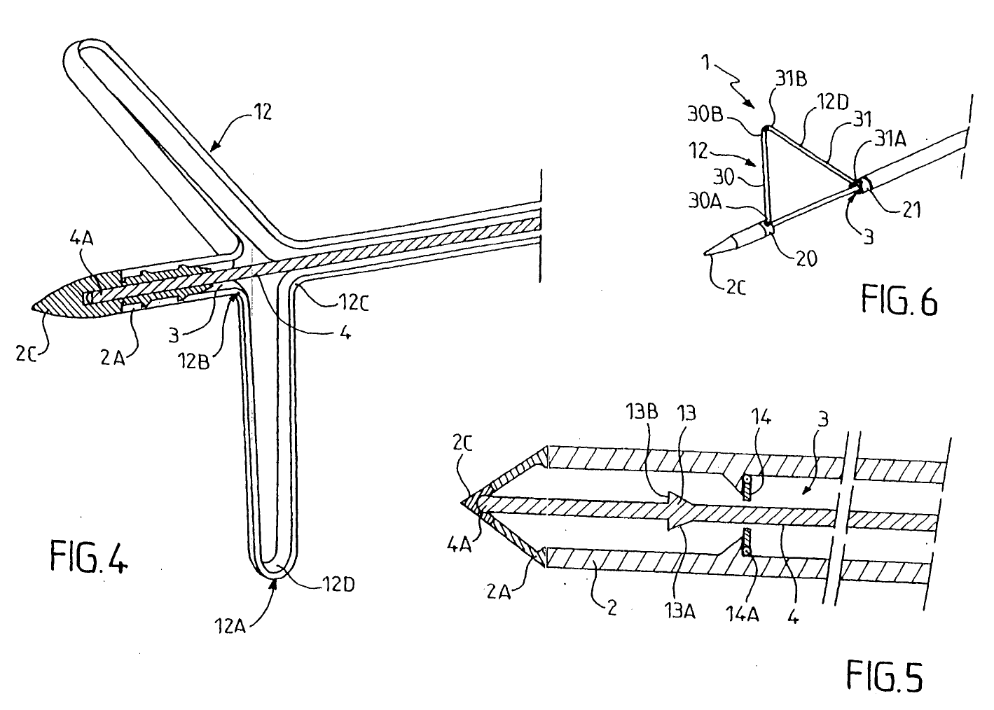

Advantageously, and as shown in FIGS. 2 and 4, the portion 11 of hollow tube 2 is made up of a plurality of beam-shaped pieces which, under the effect of axial compression, bend to a large extent to form a plurality of anchor arms 12D, naturally without breaking.

This characteristic makes the process of activating/deactivating the anchor means reversible. Thus, the explantation device of the invention can be for use once only or for use more than once, in which case it can be sterilized as many times as necessary.

Naturally, it is possible to consider working the bend zones 12A, 12B, 12C of the pieces (i.e. mainly the ends and the middle zones) so as to impart to them a predisposition to folding. It can thus be imagined that a smaller thickness of material can be provided at such zones, or else separate hinges can be installed thereat.

Advantageously, and as shown in FIGS. 1, 2, and 4, the weak segments 18 extend rectilinearly between the front section 9 and back section 10.

Also in a preferred manner, the weak segment(s) 18 extend parallel to the axis of symmetry of the hollow tube 2 between the front section 9 and the back section 10, as shown in FIGS. 1, 2, and 4.

However, it is possible, without going beyond the ambit of the invention, for the weak segment(s) 18 to extend in other shapes, e.g. in an undulating curve or undulating curves, or in zigzags between the front section 9 and the back section 10.

Advantageously, there are at least two weak segments 18 and they are angularly distributed in uniform manner.

Preferably, as shown in FIG. 2, there are three weak segments 18 which are distributed uniformly around the circumference of the hollow tube.

Advantageously, and as shown in FIG. 2, the weak segments are of identical type.

Advantageously, the weak segment(s) (18) is/are constituted by a slit or by slits, as shown in FIGS. 1, 2, and 4.

In which case, it can be understood that it is extremely easy and inexpensive to manufacture the weak segment 18 since it consists merely in pre-slitting the hollow tube 2.

It is possible, without going beyond the ambit of the invention, for the weak segment(s) 18 to be constituted by means other than slits. It is possible, in particular to imagine weak segments 18 constituted by a series of successive perforations forming one or more dashed lines of material suitable for tearing under the effect of the buckling.

Advantageously, the deformation in the portion 11 of hollow tube 2 comprises folding at least substantially at the middle zone 12A of said portion 11. In particular, when the portion 11 is made up of a plurality of pieces, each of the pieces folds at its middle, as shown in FIGS. 2 and 4.

Thus, the anchor means constituted by one or more folded pieces does not have any sharp edges, roughness, or the like, and is therefore atraumatic for the walls of the stomach.

Preferably, and as shown in FIG. 5, the perforation means 2C are deactivatable. For example, such deactivation can be made possible by means shown in FIG. 5, in which the distal portion 2A is cone-shaped having a pointed tip 2C, which tip is secured to the front end 4A of the guide wire 4, e.g. by bonding with adhesive. The distal portion 2A is made of a material that is more flexible and more ductile than the material of which the remainder of the hollow tube 2 is made, so as to be capable of retracting into the internal volume 3 when traction is exerted on the end 4B of the guide wire 4. The guide wire 4 is provided with a cam 13 having a sloping slide surface 13A and a bearing surface 13B, said cam being shaped so as to co-operate with an abutment system 14 secured to the hollow tube 2 in the following manner: when traction is exerted on the guide wire 4, it moves while entraining the tip 2C, as permitted by the flexibility of the material of which the distal portion 2A is made, towards the proximal portion 2B, thereby causing contact to be established between the sloping surface 13A and the abutment 14. Said abutment is provided with opening means 14A having a hinge function so that, under the effect of the thrust exerted by the sloping surface 13A, the abutment 14 opens to allow the cam 13 past, but once the sloping surface 13A has gone past it, it returns resiliently into a locking position, so as to come into abutment against the surface 13B. The perforation means are thus deactivated because the tip 2C is retracted into the internal volume 3 and no longer projects from the hollow tube 2, the deactivation position being locked by the cam/abutment system 13, 14 with which the hollow tube 2 and the guide wire 4 are provided.

Naturally, it is possible to imagine using other technical means, such as, for example covering the perforation means 2C with a protective sheath, for achieving the deactivatable perforation function, without going beyond the ambit of the invention.

Preferably, the external connector 5 is secured in leaktight manner to the proximal portion 2B, so that the cavity 7A and the internal volume 3 form a single volume, the guide wire 4 occupying sufficiently little space in said single volume for it to be possible to provide a space for passing a fluid sucked out via tubular end-pieces 8 in the hollow tube 2. For example, such leaktight securing can be achieved by inserting the sleeve 7 by force into the proximal portion 2B, said sleeve having a connection zone 7B advantageously provided with serration-forming means 7C co-operating with complementary shapes 7D with which the proximal portion 2B is provided so as to provide reliable and leaktight securing.

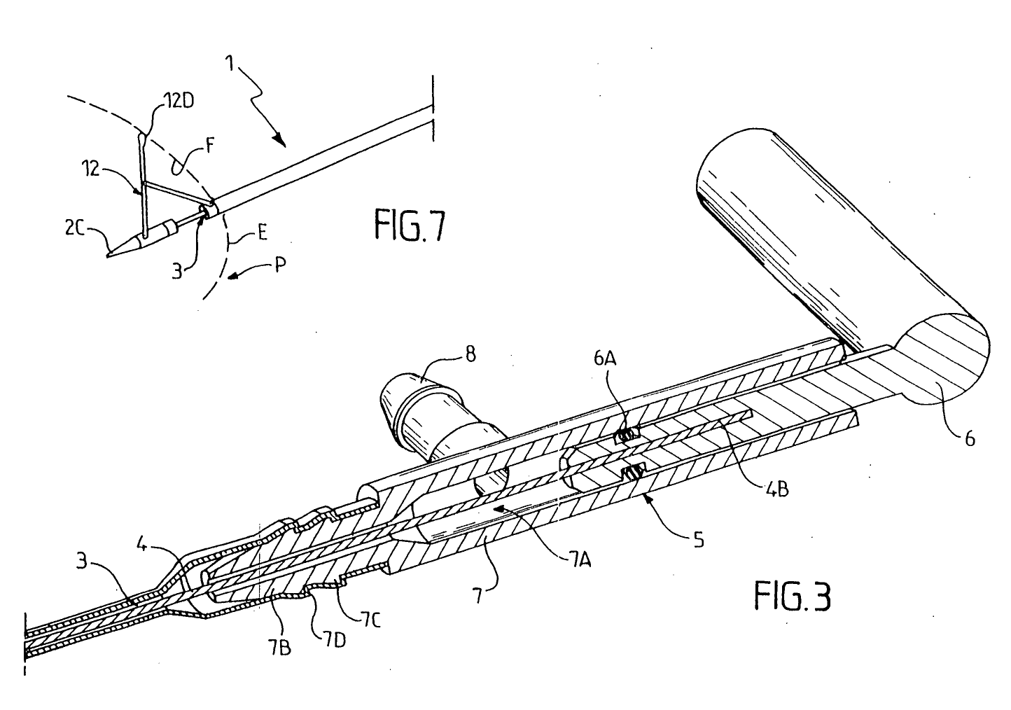

Sealing between the sleeve 7 and the control member 6 can advantageously be achieved by an O-ring seal 6A, as shown in FIG. 3.

It is thus possible, by connecting a suction instrument to the tubular end-piece 8, to suck out the fluid contained in the flexible pouch. The fluid can pass through the internal volume 3 of the hollow tube 2 via a weak segment 18 or via the opening created by deploying the anchor means 12.

Advantageously, the control member 6 is mounted to slide axially inside the cavity 7A of the sleeve 7 so that the control member 6 sliding controls traction/compression of the guide wire 4.

Naturally other modes of fixing the member 6 to the sleeve 7 are possible without going beyond the ambit of the invention.

It is thus possible to imagine mounting the control member 6 on the sleeve 7 via a pivot coupling whose axis is perpendicular to the axis of the hollow tube 2, so that the control member 6 pivoting controls the traction/compression of the guide wire 4.

It is also possible to imagine mounting the control member 6 in the cavity 7A in the sleeve via a helical translation coupling whose axis is parallel to the axis of the hollow tube 2, so that control member 6 moving (by being tightened/loosened) controls the traction/compression of the guide wire 4.

Advantageously, the medical explantation device 1 of the invention is organized so that, when the guide wire 4 is subjected to a compression force induced by action from the user on the control member 6, a portion of the guide wire 4 is capable of coming out of the internal volume 3 so as to be deployed to form a snare loop 4C, whose perimeter is adjustable by the user acting on the control member 6. The explantation device 1 thus retains the snare function that is well-known to practitioners, which can be useful in the event that it is impossible to anchor inside the balloon, e.g. if the balloon is damaged, for example. In addition, the snare as obtained with the explantation device 1 of the invention, is particularly easy to manipulate and engages particularly well, in particular since the loop 4C of the snare “bears against” a rigid structure, constituted by the hollow tube 2.

Advantageously, the medical device of the invention for explanting a flexible pouch is particularly well suited to retrieving intra-gastric balloons endoscopically from the stomach, such balloons being used to treat obesity.

In which case, the explantation device 1 of the invention operates as follows:

The explantation device 1 is inserted into the operator channel of an endoscope plunged into the digestive tube of a patient. The endoscope provided with an operator channel of diameter preferably at least equal to 2.8 mm, makes it possible, at the same time, to view the intra-gastric balloon to be extracted and to approach it directly in the stomach. The balloon can optionally be filled with a liquid or gaseous substance.

By moving the hollow tube 2 in the operator channel of the endoscope, the practitioner performing the explantation operation perforates the intra-gastric balloon by means of the deactivatable perforation means 2C, and inserts the distal portion 2A of the hollow tube 2 into the balloon at least to the section 10.

The practitioner then acts on the control member 6 so as to exert a traction force on the guide wire 4. Thus, as shown, for example, in FIG. 2, under the effect of said traction force, the hollow tube 2 is compressed axially, which, at the weak segments 8, has the effect of acting by buckling to deploy the anchor means 12, advantageously formed by a single-arm or a multi-arm anchor structure.

For a device of the type shown in FIG. 5, this operation also has the effect of deactivating the perforation means 2C.

The position of the intra-gastric balloon is thus stabilized by the anchor structure. Whereupon, the practitioner can empty the balloon, by connecting the explantation device 1 to a suction system via the end-piece 8 of the external connector 5.

Once the balloon has been emptied, the practitioner then pulls on the hollow tube 2 so as to lock the envelope E of the balloon pressed between the anchor structure and the edge of the distal orifice of the endoscope. The practitioner can then extract the balloon by exerting continuous traction on the guide wire 4 via the control member 6, so as to keep the anchor means 12 deployed, while bringing the endoscope up out of the body of the patient.

The explantation operation is thus performed with a single, atraumatic tool incorporating the functions of perforating the intra-gastric balloon, of sucking out the contents of the intra-gastric balloon, and of internally anchoring said balloon.

If the wall of the balloon tears or slips off the anchor means 12, the explantation device also makes it possible to deploy a snare loop 4C as follows: it is necessary merely to push out the guide wire 4 via the control member 6 until a loop 4C is formed via one of the openings formed in the hollow tube 2 as a result of the anchor means 12 being deployed.

It should be noted that this does not result in the perforation means 2C being reactivated because the cam 13 co-operates with the abutment 14.

The resulting snare is also atraumatic for the walls of the stomach.

The practitioner can then catch the intra-gastric balloon with the snare, which is a tool that the practitioner is used to using. The fact that the snare bears against a structure (formed by the hollow tube 2) that is more rigid than the usual structures of this type of instrument makes it possible for the catching to be more effective while manipulation is simplified. Since the perforation means 2C are deactivated, there is no risk that the practitioner might injure the patient during the operation.

Thus, the explantation device of the invention offers the practitioner greater simplicity, safety, and operating effectiveness, while also reducing the number of actions to be taken.

### SUSCEPTIBILITY OF INDUSTRIAL APPLICATION

An industrial application of the invention lies in designing, manufacturing, and using explantation devices in the medical field.

1. A medical device (1) for explanting a flexible pouch (P) containing a fluid, said pouch (P) comprises an envelope (E) having an inside face (F), said medical device comprising:
perforation means (2C) for perforating the pouch (P); and
securing means for securing the medical device to the pouch (P) so as to explant it;
wherein said perforation means (2C) make it possible to form an orifice in the flexible pouch (P) so as to enable the securing means to pass through, said securing means formed by anchor means (12) acting from the inside of the pouch (P) on a portion of said inside face (F) of the envelope to generate bearing engagement sufficient to enable the pouch to be explanted.

2. The medical device (1) for explanting a flexible pouch (P) according to claim 1, wherein the anchor means (12) are capable of being deployed, their deployment being controlled by a control member (6).

3. The medical device (1) for explanting a flexible pouch (P) according to claim 1, the medical device further comprising fluid removal means (3, 8) for removing the fluid contained inside the pouch to the outside of the body of the patient.

4. The medical device (1) for explanting a flexible pouch (P) according to claim 2, wherein the anchor means (12) are formed by a structure forming a hinged truss and comprising at least one anchor arm (12D), said structure having a front termination (20) and a back termination (21), which terminations are caused to move closer together in controlled manner by the control member (6) in order to cause the at least one anchor arm (12D) to be deployed.

5. The medical device (1) for explanting a flexible pouch (P) according to claim 2, the medical device further comprising:
a hollow tube (2) having a distal portion (2A) provided with the perforation means (2C) and a proximal portion (2B), said distal portion (2A) and said proximal portion (2B) defining between them an internal volume (3);
a guide wire (4) disposed in said internal volume (3) and having a front end (4A) and a back end (4B); and
an external connector (5) mounted at the proximal portion (2B) via a sleeve (7) having a cavity (7A), and including the control member (6) organized to make it possible for a user to exert at least traction and compression forces on the guide wire (4), said control member (6) being mounted to move relative to the sleeve (7), the cavity (7A) of said sleeve communicating with the outside via one or more tubular end-pieces (8);
said front end (4A) and said back end (4B) being secured respectively to the distal portion (2A) and to the control member (6), the hollow tube (2) including at least one weak segment (18) extending between a front section (9) and a back section (10), over a length sufficient to define with said sections (9, 10) a portion (11) of hollow tube that, when the user exerts traction on the back end (4B) via the control member (6), thereby causing the hollow tube (2) to be compressed axially, tends to undergo buckling causing it to be deformed towards the outside of the internal volume (3) along said at least one weak segment (18) so that said portion (11) forms the anchor means (12) suitable for being deployed.

6. The medical device (1) for explanting a flexible pouch (P) according to claim 5, wherein the at least one weak segment (18) extends rectilinearly between the front section (9) and the back section (10).

7. The medical device (1) for explanting a flexible pouch (P) according to claim 5, wherein the at least one weak segment (18) extends parallel to the axis of symmetry of the hollow tube (2) between the front section (9) and the back section (10).

8. The medical device (1) for explanting a flexible pouch (P) according to claim 5, wherein the at least one weak segment (18) extends in the form of an undulating curve or undulating curves, or in the form of zigzags between the front section (9) and the back section (10).

9. The medical device (1) for explanting a flexible pouch (P) according to claim 5, further comprising at least two weak segments (18) which are angularly distributed in uniform manner.

10. The medical device (1) for explanting a flexible pouch (P) according to claim 9, wherein the weak segments (18) are of identical type.

11. The medical device (1) for explanting a flexible pouch (P) according to claim 5, wherein the at least one weak segment (18) is constituted by a slit or by slits.

12. The medical device (1) for explanting a flexible pouch (P) according to claim 5, wherein the at least one weak segment (18) is constituted by a series of successive perforations forming one or more dashed lines of material suitable for tearing under the effect of the buckling.

13. The medical device (1) for explanting a flexible pouch (P) according to claim 5, wherein the portion (11) of hollow tube (2) is provided with at least one fold substantially at the middle zone (12A) of said portion.

14. The medical device (1) for explanting a flexible pouch (P) according to claim 1, wherein the perforation means (2C) are deactivatable.

15. The medical device (1) for explanting a flexible pouch (P) according to claim 5, wherein the external connector (5) is secured in leaktight manner to the proximal portion (2B), so that the cavity (7A) and the internal volume (3) form a single volume, the guide wire (4) occupying sufficiently little space in said single volume for it to be possible to provide a space for passing a fluid sucked out via tubular end-pieces (8) in the hollow tube (2).

16. The medical device (1) for explanting a flexible pouch (P) according to claim 5, wherein the control member (6) is mounted to slide axially inside the cavity (7A) of the sleeve (7) so that the control member (6), when sliding, controls traction/compression of the guide wire (4).

17. The medical device (1) for explanting a flexible pouch (P) according to claim 5, wherein the control member (6) is mounted on the sleeve (7) via a pivot coupling whose axis is perpendicular to the axis of the hollow tube (2), so that the control member (6), when pivoting, controls the traction/compression of the guide wire (4).

18. The medical device (1) for explanting a flexible pouch (P) according to claim 5, wherein the control member (6) is mounted in the cavity (7A) in the sleeve (7) via a helical translation coupling whose axis is parallel to the axis of the hollow tube (2), so that control member (6), when moving, controls the traction/compression of the guide wire (4).

19. The medical device (1) for explanting a flexible pouch (P) according to claim 5, wherein, when the guide wire (4) is subjected to a compression force induced by action from the user on the control member (6), a portion of the guide wire (4) is capable of coming out of the internal volume (3) so as to be deployed to form a snare loop (4C), whose perimeter is adjustable by the user acting on the control member (6).

20. The medical device (1) for explanting a flexible pouch (P) according to claim 1, wherein the flexible pouch is an intra-gastric balloon designed to be used in treating obesity.
